(19) United States
(12) Patent Application Publication
(10) Pub. No.: US 2010/0092887 A1
(43) Pub. Date: Apr. 15, 2010

(54) FLUORINE-CONTAINING QUINACRIDONES IN COLOUR FILTERS FOR LCDs

(75) Inventors: ULRICH FELDHUES, BERGISCH GLADBACH (DE); FRANK LINKE, KOLN (DE); STEPHAN MICHAELIS, ODENTHAL (DE); DIRK PFUETZENREUTER, BURSCHEID (DE); HORST BERNETH, LEVERKUSEN (DE); MEINHARD ROLF, LEVERKUSEN (DE)

Correspondence Address: LANXESS CORPORATION, 111 RIDC PARK WEST DRIVE, PITTSBURGH, PA, 15275-1112, US

(73) Assignee: LANXESS DEUTSCHLAND GMBH, LEVERKUSEN (DE)

(21) Appl. No.: 12/468,307

(22) Filed: May 19, 2009

(30) Foreign Application Priority Data
May 24, 2008 (DE) 102008025006.6

(51) Int. Cl. G03F 7/004; C09B 48/00; C07D 471/04; C07D 491/153

(52) U.S. Cl. 430/270.1; 106/495; 546/56; 546/57; 546/41

(57) ABSTRACT

Use of quinacridones of the formula (I)

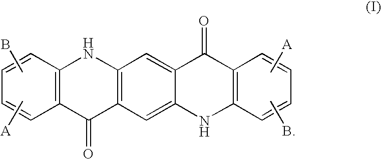

in which
A is an organic radical substituted by one or more fluorine atoms,
B is H, F, Cl, Br or an optionally substituted organic radical which may optionally together with A form a ring
   in colour filters for LCDs.

The invention relates to the use of specific fluorine-containing quinacridones in colour filters for LCDs (liquid-crystal displays) and also to formulations of such colorants and to their use for producing colour filters, to the colour filters themselves, and also to new quinacridones.

Colour filters are chiefly employed today in liquid-crystal displays and screens, colour resolution instruments and sensors. One known example are the flat screens on personal computers, televisions and video cameras. There are various ways to produce colour filters, which differ not only in the way the colours are applied but in the generation of the colour element patterns from the primary colours red, green and blue, as well as black. The colours may be applied, for example, by colouring a base layer (e.g. gelatine) by means of soluble dyes or pigments (“Dye Method”, “Dye Dispersion Method”), screenprinting, offset printing or ink-jet printing of pigment pastes, pigment formulations or pigment inks, electrodeposition of photoresists based on dyes or pigments, and also, in particular, by means of the pigment dispersion method, which uses pigments dispersed either in a polyimide resins (“non-photosensitive polyimide method”) or in a photoresist (“photosensitive acrylic method”). Associated with the stated methods, both the direct generation of the colour element patterns, by printing, and the indirect, photolithographic generation, are important, the latter in relation to the aforementioned pigment dispersion method in particular. The technique of the pigment dispersion method in the form of the “non-photosensitive polyimide method”, for example, is disclosed in JP-A-11-217514 (1998).

In the case of the pigment dispersion method involving a photoresist, the colour-imparting pigments are in fine dispersion (distribution) in a UV-curable photoresist. This photoresist, as well as the pigment, is generally composed of binder resin, polymerizable monomer, photoinitiator and, optionally, a solvent. It is prepared by, for example, first finely dispersing the pigment in the form of a concentrate in solvent and, optionally, binder resin, and adjusting the dispersion immediately prior to application together with the monomer and the photoinitiator and any further components as well. The pigmented photoresist is applied uniformly to the substrate, glass for example, by means for example of the spincoating method, and is predried, UV-exposed by means of a photomask, developed to the desired colour element patterns by means of a generally inorganic alkaline solution, and the coating is cleaned and optionally aftercured. This operation is repeated for each colour, i.e. generally three times for a trichromatism in the colours red, green and blue, for example.

The advantages associated with the use of pigments in conjunction with the pigment dispersion method lie in the improved light resistance, moisture resistance and temperature resistance of the colour filters as compared with dye-based coating systems. On the other hand, the transparency and colour purity of coatings based on pigments, irrespective of the coating method, are still unsatisfactory. Particularly when different pigments are incorporated in a mixture to shade the mixture to the desired colour locus values in a photoresist, there are unwanted losses in brilliance and transparency, with the result that operation of the displays or screens (LCDs) unavoidably entails an increased energy cost.

Red colour filters often use pigments of the perylenetetracarboxylic diimide type such as C.I. Pigment Red 179 or diketopyrrolopyrrole type such as C.I. Pigment Red 254 or anthraquinones such as C.I. Pigment Red 177.

EP-A-1004941 describes mixed crystals of quinacridones such as C.I. Pigment Violet 19 and C.I. Pigment Red 122 and their use inter alia for colour filters. Besides the specific mixed crystal, there is also a general mention of those with quinacridones substituted by fluorine on the quinacridone nucleus.

JP-A-2002348493 discloses mixtures of quinacridones optionally substituted by fluorine on the quinacridone nucleus, and their sulphonated derivatives, for colour filters. Some of these types of pigments already feature high lightfastness and colour strength. Transparency and colour purity remain unsatisfactory. Moreover, the production of mixed crystals (solid solutions) frequently entails reproducibility problems in terms of the quality, which may then have deleterious consequences for transparency and colour purity in particular, but also for colour strength and lightfastness.

The object of the present invention, accordingly, is to provide red organic pigments and their use in red colour filters for LCDs, and also formulations of such organic pigments, that do not have these disadvantages.

The invention accordingly provides for the use of quinacridones of the formula (I)

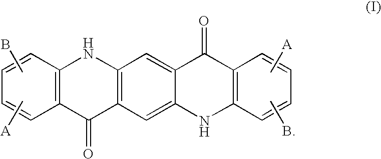

in which
A is an organic radical substituted by one or more fluorine atoms,
B is H, F, Cl, Br or an optionally substituted organic radical which may optionally together with A form a ring
   in colour filters for LCDs.

The compounds of the formula (I) preferably contain no sulpho groups.

Preferably B is hydrogen and A is C₁-C₈-alkyl, C₁-C₈-alkoxy, phenyl or phenoxy, each of which is substituted by one or more fluorine atoms.

Likewise preferably A and B together form a bridge which is substituted by one or more fluorine atoms and which with two adjacent C atoms of the benzene ring of the formula (I) forms a five-, six- or seven-membered ring which is carbocyclic or may contain heteroatoms such as O, S or N.

Suitable fluorine-substituted C₁-C₈-alkyl radicals or C₁-C₈-alkoxy radicals are, for example, methyl, ethyl or optionally branched propyl, butyl, pentyl, hexyl or octyl radicals or the corresponding alkoxy radicals which carry at least one fluorine atom. Examples are fluoromethyl, trifluoromethyl, 2-fluoroethyl, 2,2,2-trifluoroethyl, pentafluoroethyl, 2,2,3,3-tetrafluoropropyl, perfluorobutyl, perfluorooctyl, especially perfluoro-n-octyl, and the corresponding alkoxy radicals.

Suitable fluorine-substituted phenyl or phenoxy radicals are, for example, 2-, 3- or 4-fluorophenyl or -phenoxy, 2,4- or 3,4-difluorophenyl or -phenoxy, pentafluorophenyl or -phenoxy.

Suitable optionally substituted organic radicals as a possible definition of B are preferably organic radicals substituted by one or more fluorine atoms, especially those which independently of A have the same definition as the radical A. Particularly preferred are the preferred definitions of A, especially trifluoromethyl.

A suitable bridge formed by A and B together is for example —O—CF₂—O—, —O—CF₂CF₂—O—, OCHF—CHF—O— or —O—CF₂CF₂CF₂—O—.

Preferred pigments of the formula (I) correspond to the formulae (II) to (VIII).

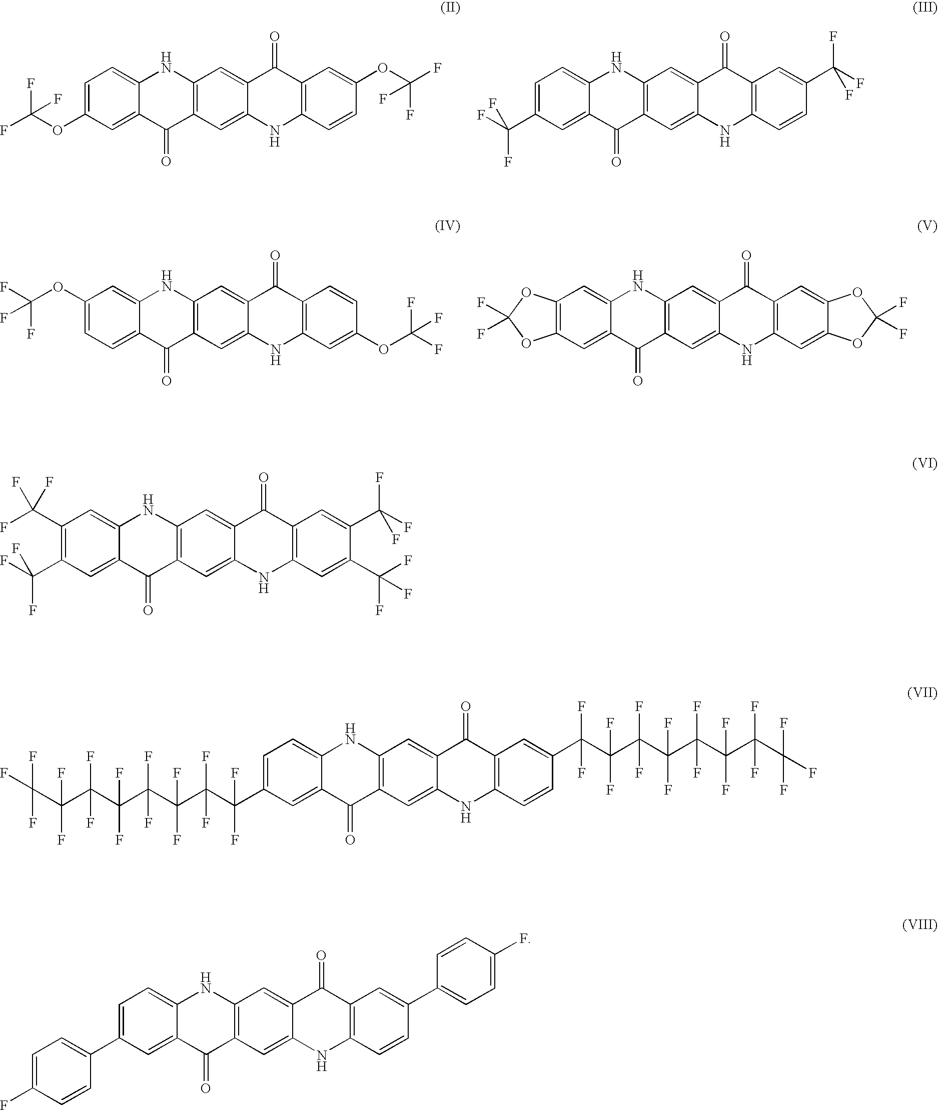

Particularly preferred are new pigments of the formula (II) which in the X-ray diffraction diagram (Cu—K_{α} radiation) exhibit lines at the following d values (d: 5.30; d: 4.09; d: 3.69; d: 3.22) and which in this specification are referred to as modification A.

Likewise particularly preferred are new pigments of the formula (II) which in the X-ray diffraction diagram (Cu—K_{α} radiation) exhibit lines at the following d values (d: 4.22; d: 3.55; d: 3.31) and which in this specification are referred to as modification B.

The particle size and surface area of the pigments used in accordance with the invention can be adjusted by methods which are known per se and are set out, for example, in U.S. Pat. No. 6,068,695, such as salt kneading or ball milling and/or optionally downstream finishing steps such as, for example, heat treatments in aqueous, organic or aqueous/organic solvents with or without addition of additives.

The pigments used in accordance with the invention preferably possess a specific surface area of 40 to 200 m²/g, in particular of 60 to 140 m²/g, very preferably of 70 to 120 m²/g. The surface area is determined in accordance with DIN 66131: Determination of the specific surface area of solids by gas adsorption by the method of Brunauer, Emmett and Teller (B.E.T.).

The pigments used in accordance with the invention preferably possess a dispersion harshness of 10 to 500, measured according to DIN 53775, part 7, the temperature of cold rolling being 25° C. and the temperature of hot rolling being 150° C. Pigments used with particular preference possess a dispersion harshness of 20 to 250.

The dispersion harshness is measured in accordance with DIN 53 775, part 7, the temperature of cold rolling being 25° C. and that of hot rolling being 150° C.

All of the dispersion harshnesses reported in this specification were determined in accordance with this modified DIN specification.

The pigments used in accordance with the invention preferably possess a particle size (longitudinal axis in the transmission electron microscope) of 10 to 200 nm, in particular of 20 to 100 nm. Preferably the pigments used in accordance with the invention possess a narrow particle size distribution with a relative standard deviation (standard deviation/particle size)<50%, especially <35%, more preferably <20%. Preferably the pigments used in accordance with the invention possess a length-to-width ratio of 5:1 to 1:1, in particular of 3:1 to 1:1, more preferably of 2:1 to 1.2:1.

The pigments of the formula (I) used in accordance with the invention can also be employed in combination with other pigments, for the purpose, for example, of optimizing the optical properties of the colour filters. The invention does not impose any restriction on the selection of other pigments for possible additional use. Both organic and inorganic pigments are suitable.

Preferred organic pigments are, for example, those of the monoazo, disazo, laked azo, β-naphthol, Napthol AS, benzimidazolone, quinacridone, disazo condensation, azo metal complex, isoindoline and isoindolinone series, and also polycyclic pigments such as, for example, from the phthalocyanine, quinacridone (other than those of the formula I), perylene, perinone, thioindigo, anthraquinone, dioxazine, quinophthalone and diketopyrrolopyrrole series. In addition, laked dyes, especially Ca, Mg and Al lakes of dyes containing sulphonic or carboxylic acid groups. With very particular preference, the melamine-intercalated nickel complex of azobarbituric acid is claimed as a pigment for accompanying use.

Examples of other organic pigments which are intended for optionally accompanying use and which are known in the Colour Index are:

Colour Index Pigment Yellow 12, 13, 14, 17, 20, 24, 74, 83, 86, 93, 94, 109, 110, 117, 125, 137, 138, 139, 147, 148, 150, 153, 154, 166, 173, 185,

Colour Index Pigment Orange 13, 31, 36, 38, 40, 42, 43, 51, 55, 59, 61, 64, 65, 71, 72, 73,

Colour Index Pigment Red 9, 97, 122, 123, 144, 149, 166, 168, 177, 179, 180, 192, 215, 216, 224, 254, 272,

Colour Index Pigment Green 7, 10, 36, 37, 45,

Colour Index Pigment Blue 15, 15:1, 15:2, 15:3, 15:4, 15:6, 16,

Colour Index Pigment Violet 19, 23.

Further pigments not known from the Colour Index are, for example, the melamine-intercalated nickel-azobarbituric acid complex pigment of the formula

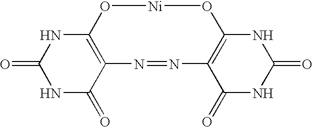

and also its tautomeric forms, known from DE102005033581, especially Example 2.

Mention may likewise be made of sulphonated derivatives of the pigments of the formula (I) used in accordance with the invention.

Where “other pigments”—other than those of the formula (I)—are used additionally, the fraction of “pigment” as defined above, conforming to the formula (I), is preferably 1-99% by weight, in particular 20-80% by weight, based on the total amount employed of all pigments.

Pigments preferred for accompanying use are Pigment Red 122, Pigment Red 149, Pigment Red 177, Pigment Red 179, Pigment Red 254, Pigment Violet 19, Pigment Yellow 138, Pigment Yellow 139, Pigment Yellow 150 or a melamine-intercalated nickel-azobarbituric acid complex pigment.

Particular preference is given to the use of pigment mixtures comprising
at least one pigment of the formula (I)
at least one further red pigment from the group consisting of C.I. Pigment Red 122, C.I. Pigment Red 149, C.I. Pigment Red 177, C.I. Pigment Red 179, C.I. Pigment Red 254 and C.I. Pigment Violet 19

in colour filters for LCDs.

This mixture for preferred use optionally comprises additionally one or more yellow pigments.

The yellow or orange pigments used accompanyingly preferably possess an absorption band in the range from 400 to 520 nm.

In particular this preferred mixture comprises yellow pigments selected from the group consisting of C.I. Pigment Yellow 138, C.I. Pigment Yellow 139, C.I. Pigment Yellow 150 and a melamine-intercalated nickel-azobarbituric acid complex pigment.

The mixtures per se are likewise provided by this invention.

Where yellow or orange pigments are used as “other pigments”, the fraction of these yellow or orange “other pigments” is preferably 1% to 50% by weight, in particular 5%-30% by weight, based on the total amount employed of all pigments.

Where red pigments are used as “other pigments”, the fraction of these red “other pigments” is preferably 1 to 99% by weight, in particular 20%-80% by weight, based on the total amount employed of all pigments.

The colour filters produced with the pigments of the invention or mixtures thereof are notable in particular for high colour purity and excellent transparency.

Likewise with preference the pigments of the formula (I) are used in the form of a mixture which besides the formula (I) also comprises yellow pigments. Preferably those which possess an absorption band in the range from 400 to 520 nm. In particular this preferred mixture comprises yellow pigments selected from the group consisting of C.I. Pigment Yellow 138, C.I. Pigment Yellow 139, C.I. Pigment Yellow 150 and a melamine-intercalated 1:1 nickel-azobarbituric acid complex pigment. This mixture per se as well is likewise provided by this invention.

The inventive use of the above-described pigments or pigment mixtures for producing colour filters for liquid-crystal displays will be described below with reference to the example of the pigment dispersion method according to the photoresist method.

The inventive use of the pigments of the invention for producing colour filters is characterized for example in that the pigment, optionally with a binder resin and an organic solvent, optionally with addition of a dispersant, is homogenized and then subjected to continuous or batchwise wet-comminution, in particular to a particle size by number (electron-microscopic determination) of 99.5%<1000 nm, preferably 95%<500 nm and in particular 90%<200 nm. Suitable wet-comminution methods include, for example, stirrer or dissolver dispersion, grinding by means of stirred ball mills or bead mills, kneaders, roll mill, high-pressure homogenization or ultrasonic dispersion.

The dispersing treatment is accompanied or followed by the addition of at least one photocurable monomer and a photoinitiator. Following dispersion, further binder resin, solvent or customary photoresist adjuvants may be introduced as is necessary for the desired photosensitive coating formulation (photoresist) for producing the colour filters. For the purposes of this invention, a photoresist is a formulation comprising at least one photocurable monomer and a photoinitiator in addition to the formula (I) pigment.

Useful dispersants include generally commercially available dispersants, such as polymeric, ionic or nonionic dispersants, based for example on polycarboxylic acids or polysulphonic acids, and also polyethylene oxide-polypropylene oxide block copolymers. Derivatives of organic dyes can also be used, furthermore, as dispersants or co-dispersants.

The production of colour filters therefore gives rise to “preparations” which comprise, based on the preparation:
at least one quinacridone of the formula (I), referred to for the purposes of this specification as pigment of the invention,
optionally one or more other pigments,
optionally a binder resin,
at least one organic solvent, and
optionally a dispersant.

In one preferred embodiment the preparation contains (amounts based on preparation):

1-50% by weight of at least one quinacridone of the formula (I)

0-50% by weightof one or more other pigments

0-20% by weight of binder resins

0-20% by weight of dispersants

10-94% by weight of organic solvent.

The coating of the photoresist onto a plate to produce the coloured image element pattern can be carried out by either direct or indirect application. Examples of application methods that may be mentioned include ink jet, roller coating, spincoating, spray coating, dip coating and air knife coating.

Examples of suitable plates include, depending on use, the following: transparent glasses such as white or blue glass plate, silicate-coated blue glass plate, synthetic resin plate or synthetic resin films based for example on polyester resin, polycarbonate resin, acrylic resin or vinyl chloride resin, and additionally metal plates based on aluminum, copper, nickel or steel, and also ceramic plates or semiconductor plates with photoelectric transfer elements applied.

Application is generally effected in such a way that the photosensitive layer obtained is 0.1 to 10 μm thick.

Application may be followed by thermal drying of the layer.

Exposure takes place preferably by exposing the photosensitive layer to an active light beam in the form, preferably, of an image pattern by means of photomask. This cures the layer at the exposed areas. Examples of suitable light sources include the following: high-pressure and ultrahigh-pressure mercury vapour lamp, xenon lamp, metal halide lamp, fluorescent lamp, and laser beam in the visible region.

Development following exposure removes the unexposed portion of the coating, to give the desired image pattern form of the colour elements. Customary development methods include spraying with or dipping in aqueous alkaline developer solution or in an organic solvent that contains inorganic alkali such as, for example, sodium hydroxide or potassium hydroxide, sodium metasilicate or organic bases such as monoethanolamine, diethanolamine, triethanolamine, triethylamine or salts thereof.

Development is generally followed by thermal afterdrying/-curing of the image patterns.

As binder resins which can be used together with the “pigment” or pigment formulations based thereon (i.e. containing binder resin and pigment of the formula (I)) in colour filters or in the preparations for producing colour filters by, for example, the pigment dispersion method, the invention imposes no particular restriction; conventional film-forming resins in particular are suitable for application in colour filters.

By way of example, binder resins from the group of the cellulose resins such as carboxymethylhydroxyethylcellulose and hydroxyethylcellulose, acrylic resins, alkyd resins, melamine resins, epoxy resins, polyvinyl alcohols, polyvinylpyrrolidones, polyamides, polyamide-imines and polyimides are suitable.

Suitable binder resins also include those containing photopolymerizable, unsaturated binders. The binder resins may for example be resins from the group of the acrylic resins. Mention may be made in particular of homopolymers and copolymers of polymerizable monomers such as methyl(meth)acrylate, ethyl (meth)acrylate, propyl(meth)acrylate, butyl(meth)acrylate, styrene and styrene derivatives, and additionally copolymers between carboxyl-bearing polymerizable monomers such as (meth)acrylic acid, itaconic acid, maleic acid, maleic anhydride, maleic acid monoalkyl esters, particularly with alkyl of 1 to 12 carbon atoms, and polymerizable monomers such as (meth)acrylic acid, styrene and styrene derivatives, such as α-methylstyrene, m- or p-methoxystyrene, p-hydroxystyrene, for example. Examples that may be mentioned are reaction products of carboxyl-containing polymeric compounds with compounds which contain in each case one oxirane ring and an ethylenically unsaturated compound such as, for example, glycidyl(meth)acrylate, acryloyl glycidyl ether and itaconic acid monoalkylglycidyl ethers, etc., and also reaction products of carboxyl-containing polymeric compounds with compounds containing one hydroxyl group and an ethylenically unsaturated bond (unsaturated alcohols), such as allyl alcohol, 2-buten-4-ol, oleyl alcohol, 2-hydroxyethyl(meth)acrylate, N-methylolacrylamide, etc; binder resins of this kind may further comprise unsaturated compounds which possess free isocyanate groups.

In general the equivalence of the unsaturation (molar weight of binder resin per unsaturated compound) of the said binder resins is 200 to 3000, in particular 230 to 1000, to provide not only adequate photopolymerizability but also film hardness. The acid value is generally 20 to 300, in particular 40 to 200, to provide sufficient alkali developability following exposure of the film.

The average molar weight of the binder resins to be used is between 1500 and 200 000, in particular 10 000 to 50 000 g/mol.

The organic solvents used in the context of the inventive use of the pigment formulations for colour filters are, for example, ketones, alkylene glycol ethers, alcohols and aromatic compounds. Examples are, from the group of the ketones: acetone, methyl ethyl ketone, cyclohexanone, etc.; from the group of the alkylene glycol ethers: methylcellosolve (ethylene glycol monomethyl ether), butylcellosolve (ethylene glycol monobutyl ether), methylcellosolve acetate, ethylcellosolve acetate, butylcellosolve acetate, ethylene glycol monopropyl ether, ethylene glycol monohexyl ether, ethylene glycol dimethyl ether, diethylene glycol ethyl ether, diethylene glycol diethyl ether, propylene glycol monomethyl ether, propylene glycol monoethyl ether, propylene glycol monopropyl ether, propylene glycol monobutyl ether, propylene glycol monomethyl ether acetate, diethylene glycol methyl ether acetate, diethylene glycol ethyl ether acetate, diethylene glycol propyl ether acetate, diethylene glycol isopropyl ether acetate, diethylene glycol butyl ether acetate, diethylene glycol tert-butyl ether acetate, triethylene glycol methyl ether acetate, triethylene glycol ethyl ether acetate, triethylene glycol propyl ether acetate, triethylene glycol isopropyl ether acetate, triethylene glycol butyl ether acetate, triethylene glycol tert-butyl ether acetate, etc.; from the group of the alcohols: methyl alcohol, ethyl alcohol, isopropyl alcohol, n-butyl alcohol, 3-methyl-3-methoxybutanol, etc.; and, from the group of the aromatic solvents, benzene, toluene, xylene, N-methyl-2-pyrrolidone, ethyl N-hydroxymethyl-2-acetate, etc.

Further other solvents are for example 1,2-propanediol diacetate, 3-methyl-3-methoxybutyl acetate, ethyl acetate, tetrahydrofuran, etc. The solvents can be used individually or in mixtures with one another.

The invention further provides a photoresist comprising at least one pigment as defined above or at least one pigment formulation of the invention and at least one photocurable monomer and also at least one photoinitiator.

The photocurable monomers contain at least one reactive double bond and optionally other reactive groups in the molecule.

Photocurable monomers may be interpreted in this context as being, in particular, reactive solvents or what are called reactive diluents from the group, for example, of the mono-, di-, tri- and multifunctional acrylates and methacrylates, vinyl ethers, and glycidyl ethers. Suitable reactive groups additionally present include allyl, hydroxyl, phosphate, urethane, secondary amine and N-alkoxymethyl groups. Monomers of this kind are known to the skilled person and are listed for example in [Römpp Lexikon, Lacke und Druckfarben, Dr. Ulrich Zorll, Thieme Verlag Stuttgart-New York, 1998, pp. 491/492] or online at http://www.roempp.com under the entry heading ‘Reaktivverdünner’ [Reactive diluents]

The selection of the monomers is guided in particular by the nature and intensity of the exposing radiation used, the desired reaction with the photoinitiator, and the film properties. Monomer combinations can also be used.

Photoreaction initiators or photoinitiators may be understood as being compounds which by absorbing visible or ultraviolet radiation form reactive intermediates that are capable of inducing a polymerization reaction on the part, for example, of the abovementioned monomers and/or binder resins. Photoreaction initiators are likewise general knowledge and may likewise be taken from [Römpp Lexikon, Lacke und Druckfarben, Dr. Ulrich Zorll, Thieme Verlag Stuttgart-New York, 1998, pp. 445/446] or online at http://www.roempp.com under the entry heading ‘Photoinitiatoren’ [Photoinitiators].

The invention imposes no restriction with regard to the photocurable monomers or photoinitiators that are to be employed.

The invention preferably provides photoresists comprising
A) at least one “pigment” as defined above, in particular in a mixture with other pigments, or a pigment formulation of the invention that is based thereon,
B1) at least one photocurable monomer,
B2) at least one photoinitiator,
C1) optionally an organic solvent,
D) optionally a dispersant,
E) optionally a binder resin,

and optionally further additions.

The invention also imposes no restriction with regard to the technology for generating the coloured image element patterns on the basis of the solid pigment formulations or pigments for use in accordance with the invention. In addition to the above-described photolithographic method, other methods such as offset printing, chemical milling or ink-jet printing are also suitable. The selection of suitable binder resins and solvents or pigment vehicles, and of further additions, should be conformed to the particular method. In the case of the ink-jet method, which comprehends not only thermal but also mechanical and piezomechanical ink-jet printing, suitable vehicles for the pigments and optionally binder resins include not only purely organic vehicles but also aqueous-organic vehicles; aqueous-organic vehicles are in fact preferred, with suitable organic solvents being those specified above.

The invention further provides compounds of the formula (IVa)

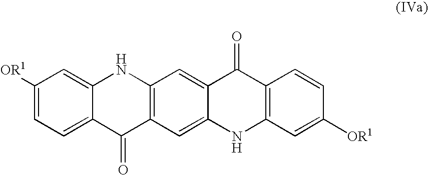

in which R¹ is a fluorine-containing aliphatic radical, especially a fluorine-containing C₁-C₈-radical, preferably fluoromethyl, trifluoromethyl, 2-fluoroethyl, 2,2,2-trifluoroethyl, pentafluoroethyl, 2,2,3,3-tetrafluoropropyl, perfluorobutyl or perfluorooctyl.

The invention further provides a process for preparing compounds of the formula (IVa), characterized in that an aniline substituted preferably in 3 position by OR¹, especially an aniline of the formula (IVb)

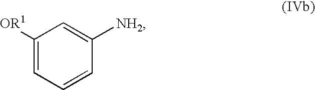

in which R¹ is a fluorine-containing aliphatic radical, especially a fluorine-containing C₁-C₈-radical, preferably fluoromethyl, trifluoromethyl, 2-fluoroethyl, 2,2,2-trifluoroethyl, pentafluoroethyl, 2,2,3,3-tetrafluoropropyl, perfluorobutyl or perfluorooctyl, is reacted with dimethyl succinylsuccinate preferably in the presence of a solvent and acidic catalyst, and the product is then oxidized and hydrolysed, and subsequently the resulting dicarboxylic acid, preferably following isolation and drying, is condensed in sulphuric acid or polyphosphoric acid to give the quinacridone.

The invention further provides compounds of the formula (Va)

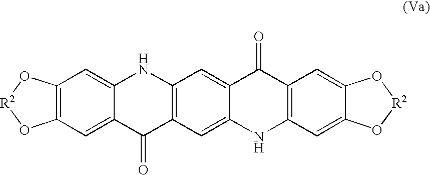

in which

R² is a fluorine-substituted C₁-C₃-alkylene unit, especially —CF₂—, —CF₂—CF₂—, —CHF—CHF— or —CF₂—CF₂—CF₂—.

The invention further provides a process for preparing compounds of the formula (Va), characterized in that an aniline fused preferably in 3,4-position with —O—R²—O—, especially an aniline of the formula (Vb)

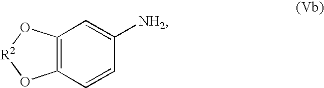

in which R² is a fluorine-substituted C₁-C₃-alkylene unit, especially —CF₂—, —CF₂—CF₂—, —CHF—CHF— or —CF₂—CF₂—CF₂—, is reacted with dimethyl succinylsuccinate, preferably in the presence of a solvent and acidic catalyst, and the product is then oxidized and hydrolysed, and subsequently the resulting dicarboxylic acid, preferably after isolation and drying, is condensed in sulphuric acid or polyphosphoric acid to give the quinacridone.

### EXAMPLES

### Example 1

a) Intermediate

1000 g of methanol were introduced, 1.00 mol of p-trifluoromethoxyaniline was added. 0.44 mol of DMSS (dimethyl succinylsuccinate) and 6 g of 98% sulphuric acid were introduced. Reaction took place at 85° C. for 2 hours under pressure. Subsequently, at room temperature, 0.44 mol of m-nitrobenzenesulphonic acid Na salt was introduced. Thereafter 2.05 mol of 50% potassium hydroxide solution were added dropwise and 37 g of water. Reaction took place at 85° C. for 3 h under pressure.

Additionally, dilution was carried out with 1500 g of water, the pH was adjusted to 4 using phosphoric acid (85% strength) and the mixture was stirred for a further hour, the product being isolated, washed and dried.

Yield: 95% dicarboxylic acid of the formula

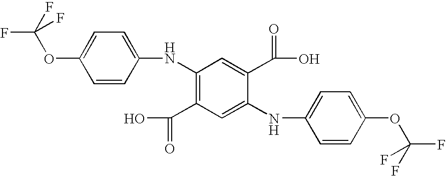

b) Cyclization

450 g of polyphosphoric acid (purity: 84% P₂O₅) were introduced and heated to about 100° C. 0.13 mol of dicarboxylic acid, prepared in Example 1, was introduced over the course of 30 min. Subsequent stirring took place at 105° C. for 2 hours. Over 2.5 hours, 375 g of 75% phosphoric acid were added dropwise at 105° C.

Over the course of 30 min, the viscous suspension was added dropwise to a charge of 500 g of water, followed by stirring for 30 min. The product was isolated, washed free of salt, then suspended in 1000 g of 2% potassium triphosphate solution, isolated again and washed to neutrality. The filtercake was triterated with methanol and then washed free of methanol with water and dried.

Yield: 91% of trifluoromethoxyquinacridone (formula II) crystal modification A

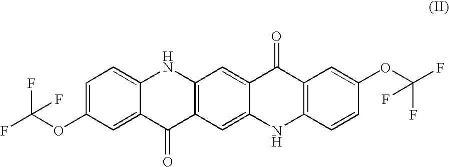

BET: 14 m²/g

c) Production of the PVC Sample for Determining the Hue and the Dispersion Harshness

For determining the dispersion harshness, 100 g of PVC paste, prepared from 4.2 parts of Vestolit® E 7004 (emulsion-PVC powder), 1.8 parts of diisooctylphthalate, 0.15 part of Baerostab® UBZ 770 (liquid barium-zinc stabilizer) and 0.125 part of Moltopren® white paste RUN 01 (pigment formulation containing 50% TiO₂) were applied at 150° C. to a Collin laboratory mixing roll mill. The roll nip was 0.8 mm.

0.1 g of the sample was applied to the PVC sheet and the roll nip was set to 0.12 mm. The rolled sheet was taken off and applied again. This operation was repeated eight times. The roll nip was set to 0.8 mm and the rolled sheet was removed. A test specimen measuring 60×60 mm was punched from the rolled sheet.

The remainder of the sheet was then applied to the roll mill at 25° C. The roll nip was 0.2 mm. The rolled sheet was taken off and applied again. This process was repeated 15 times. The sheet, which is no longer smooth from the cold rolling, was applied to the roll at 150° C. with a roll nip of 0.8 mm. After 60 s, the sheet was removed and a test specimen measuring 60×60 mm was punched from it. The rotational speed of the roll was held at a constant 20 rpm and the friction at 1:1.1.

The dispersion harshness is the percentage increase in colour strength after rolling at 25° C.

Dispersion harshness: 78

The transparent test specimens were produced in the same way, but using a test paste consisting of 4.2 parts of Vestolit® E 7004 (E-PVC powder), 1.8 parts of diisooctyl phthalate and 0.15 part of Baerostab® UBZ 770 (liquid barium-zinc stabilizer).

The hues were determined using a Gretag Macbeth spectrometer. The samples were subjected to measurement with a 10° observer angle, D65 illuminant, without a gloss track: Reflection measurements:

L*: 65.82 a*: 38.34 b*: −14.39 C*: 40.95 h°: 339.44

### Example 2

110 g of 98% sulphuric acid were introduced at 20° C. 14.9 g of trifluoromethoxyquinacridone (prepared in Example 1) were introduced over the course of 30 min at 10-25° C. The solution was diluted with 20 g of sulphuric acid and over the course of 20 min was added dropwise to 500 ml of methanol at about 20° C. The suspension was diluted with 800 g of water and the product was isolated, washed to neutrality and dried.

Yield: 13 g of trifluoromethoxyquinacridone (formula II) crystal modification B

BET: 46 m²/g

Production of the PVC sample for determining the hue and the dispersion harshness same as for Example 1c

Dispersion harshness: 600

L*: 63.37 a*: 43.64 b*: −17.11 C*: 46.87 h°: 338.59

### Example 3

370 g of polyphosphoric acid (purity: 84% P₂O₅) were introduced and heated to about 100° C. 37.2 g of dicarboxylic acid, prepared in Example 1a), were introduced over the course of 30 min. This was followed by stirring at 105° C. for 1 hour. The viscous solution was added dropwise to 500 ml of methanol at 60° C. This was followed by stirring at 60° C. for an hour and the product was then isolated, washed with water and dried.

Yield: 31.6 g of trifluoromethoxyquinacridone (formula II) crystal modification B

BET: 47 m²/g

Production of the PVC sample for determining the hue and the dispersion harshness same as for Example 1c

Dispersion harshness: 106

L*: 64.58 a*: 43.29 b*: −14.82 C*: 45.76 h°: 341.11

### Example 4

1200 g of 98% strength sulphuric acid were introduced. 150 g of trifluoromethoxyquinacridone (prepared in Example 1) were introduced over the course of 30 min at 10-25° C. and clarified via a glass frit.

1000 ml of water were introduced, 1500 g of ice were introduced. Over the course of 30 minutes, the sulphuric acid solution was added dropwise, the temperature being held at about −5° C. by addition of approximately 2500 g of ice. This was followed by stirring for about 30 minutes, after which the product was isolated and washed free of salt.

Yield: 146 g of trifluoromethoxyquinacridone (formula II) crystal modification B

BET: 72 m²/g

c) Production of the PVC sample for determining the hue and the dispersion harshness same as for Example 1c

Dispersion harshness: 20

L*: 66.57 a*: 43.40 b*: −12.91 C*: 45.28 h°: 343.44

### Example 5

Compound (IV) was prepared (starting from m-trifluoromethoxyaniline) in the same way as for Example 1, and subjected to testing.

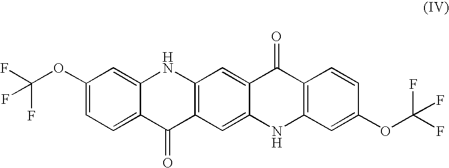

BET: 46 m²/g

Dispersion harshness: 100

L*: 64.85 a*: 30.28 b*: −1.16 C*: 30.30 h°: 357.81

### USE EXAMPLES

Preparation of a red formulation and its use for producing a red colour filter

### Use Example 1 (Not Inventive)

Pigment used: Pigment Red 122

In a stirred vessel 774 parts by weight of methoxybutyl acetate and 286 parts by weight of a 21% strength solution of an alkali-soluble copolymer (binder resin) based on benzyl methacrylate (70 parts)/2-hydroxyethyl methacrylate (15 parts)/methacrylic acid (15 parts), molar weight around 25 000 g/mol, in methoxypropyl acetate were mixed homogeneously.

Subsequently 100 parts by weight of Pigment Red 122, dried beforehand at 70° C. to a residual moisture content of less than 1% by weight, were introduced homogeneously.

This pigment suspension was ground in a horizontal, sealed bead mill using yttrium-stabilized zirconium oxide beads (diameter 0.6 to 1.0 mm) in a number of passes until an effective particle diameter (measured with laser scattering light correlation spectroscopy on an approximately 0.5% by weight dilution in methoxypropyl acetate) of less than 150 nm was obtained in conjunction with a polydispersity of less than 0.14. (For comparison, a dried film of a 1% dilution in methoxypropyl acetate was observed under electron microscopy to have a very narrow particle size distribution, with 95% of the number of particles below 100 nm.)

Preparation of a Photoresist

Introduced homogeneously with stirring into 1000 parts by weight of the resulting preparation were 34.5 parts by weight of trimethylolpropane triacrylate (monomeric reactive diluent) and 13.8 parts by weight of a photoreaction initiator based on benzophenone and N,N′-tetraethyl-4,4′-diaminobenzophenone in a ratio of 3/1 parts by weight.

This gave a UV-curable photoresist, which was applied to a transparent substrate and developed to give the colour filter.

For this purpose the photoresist was spin-coated into a section of cleaned borosilicate glass (Corning® 7059, Owens Corning Corp.) measuring 300×350 mm and was dried at 110° C. for 5 minutes in an oven under clean conditions to give a film approximately 1.5-2 μm thick.

The film, after cooling, was then UV-exposed at a dose of 200 mJ/cm² with an ultra-high-pressure mercury vapour lamp, by means of a negative mask to obtain the desired stripe image pattern, and then developed by means of 0.06% strength aqueous potassium hydroxide solution at room temperature, cleaned with fully demineralized water and dried. This was followed by a 30-minute aftercure at 235° C. in an oven under clean conditions.

### Use Example 2 (Inventive)

Pigment used: Pigment from Example 3

In a stirred vessel 774 parts by weight of methoxybutyl acetate and 286 parts by weight of a 21% strength solution of an alkali-soluble copolymer (binder resin) based on benzyl methacrylate (70 parts)/2-hydroxyethyl methacrylate (15 parts)/methacrylic acid (15 parts), molar weight around 25 000 g/mol, in methoxypropyl acetate were mixed homogeneously.

Subsequently 100 parts by weight of pigment from Example 3, dried beforehand at 70° C. to a residual moisture content of less than 1% by weight, were introduced homogeneously.

This pigment suspension was ground in a horizontal, sealed bead mill using yttrium-stabilized zirconium oxide beads (diameter 0.6 to 1.0 mm) in a number of passes until an effective particle diameter (measured with laser scattering light correlation spectroscopy on an approximately 0.5% by weight dilution in methoxypropyl acetate) of less than 150 nm was obtained in conjunction with a polydispersity of less than 0.14. (For comparison, a dried film of a 1% dilution in methoxypropyl acetate was observed under electron microscopy to have a very narrow particle size distribution, with 95% of the number of particles below 100 nm.)

Preparation of a Photoresist

Introduced homogeneously with stirring into 1000 parts by weight of the resulting preparation were 34.5 parts by weight of trimethylolpropane triacrylate (monomeric reactive diluent) and 13.8 parts by weight of a photoreaction initiator based on benzophenone and N,N′-tetraethyl-4,4′-diaminobenzophenone in a ratio of 3/1 parts by weight.

This gave a UV-curable photoresist, which was applied to a transparent substrate and developed to give the colour filter.

For this purpose the photoresist was spin-coated into a section of cleaned borosilicate glass (Corning® 7059, Owens Corning Corp.) measuring 300×350 mm and was dried at 110° C. for 5 minutes in an oven under clean conditions to give a film approximately 1.5-2 μm thick.

The film, after cooling, was then UV-exposed at a dose of 200 mJ/cm² with an ultra-high-pressure mercury vapour lamp, by means of a negative mask to obtain the desired stripe image pattern, and then developed by means of 0.06% strength aqueous potassium hydroxide solution at room temperature, cleaned with fully demineralized water and dried. This was followed by a 30-minute aftercure at 235° C. in an oven under clean conditions.

The resulting red inventive colour filter 2, produced in accordance with Use Example 2, on the basis of Example 3, possessed a significantly improved spectral transparency as compared with the non-inventive colour filter 1, produced in accordance with Use Example 1 on the basis of Pigment Red 122. The colour purity and brilliance of colour filter 2 are excellent.

### Use Example 3 (Inventive)

Pigment used: 80% pigment from Example 3
20% melamine-intercalated 1:1 nickel-azobarbituric acid pigment

Preparation of a red formulation and its use for producing a red colour filter

By the same method as described in Use Example 2, but using 80 parts by weight of the pigment from Example 3 and 20 parts by weight of a melamine-intercalated nickel-azobarbituric acid complex (prepared according to Example 2 from DE 10 2005 033 581 B4), a preparation was prepared which is very well suited to the preparation of red photoresists for colour filters.

A photoresist prepared as described in Use Example 2, and a red inventive colour filter produced using it, possessed very good spectral transparency properties and also excellent colour purity and brilliance.

### Use Example 4 (Inventive)

Pigment used: 40% pigment from Example 3
40% C.I. Pigment Red 254
20% melamine-intercalated 1:1 nickel-azobarbituric acid pigment

Preparation of a red formulation and its use for producing a red colour filter

By the same method as described in Use Example 2, but using 40 parts by weight of the pigment from Example 3, 40 parts by weight of C.I. Pigment Red 254 and 20 parts by weight of a melamine-intercalated nickel-azobarbituric acid complex (prepared according to Example 2 from DE 10 2005 033 581 B4), a preparation was prepared which is very well suited to the preparation of red photoresists for colour filters.

A photoresist prepared as described in Use Example 2, and a red inventive colour filter produced using it, possessed very good spectral transparency properties and also excellent colour purity and brilliance.

### Use Example 5 (Inventive)

Pigment used: Pigment from Example 4

Preparation of a red formulation and its use for producing a red colour filter

By the same method as described in Use Example 2, but using the pigment from Example 4, a preparation was prepared which is very well suited to the preparation of red photoresists for colour filters.

A photoresist prepared as described in Use Example 2, and a red inventive colour filter produced using it, possessed very good spectral transparency properties and also excellent colour purity and brilliance.

### Use Example 6 (Inventive)

Pigment used: Pigment from Example 5

Preparation of a red formulation and its use for producing a red colour filter

By the same method as described in Use Example 2, but using the pigment from Example 5, a preparation was prepared which is very well suited to the preparation of red photoresists for colour filters.

A photoresist prepared as described in Use Example 2, and a red inventive colour filter produced using it, possessed very good spectral transparency properties and also excellent colour purity and brilliance.

### Use Example 7 (Inventive)

Pigment used: Pigment of the formula (III)

Preparation of a red formulation and its use for producing a red colour filter

By the same method as described in Use Example 2, but using a pigment of the formula (III), a preparation was prepared which is very well suited to the preparation of red photoresists for colour filters.

A photoresist prepared as described in Use Example 2, and a red inventive colour filter produced using it, possessed very good spectral transparency properties and also excellent colour purity and brilliance.

1. A method of pigmenting color filters for liquid-crystal displays comprising pigmenting the color filters with a pigment comprising a quinacridone of the formula (I) 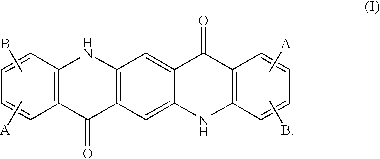
in which
A is an organic radical substituted by one or more fluorine atoms,
B is H, F, Cl, Br or an unsubstituted or substituted organic radical which may or may not together with A form a ring.

2. The method according to claim 1, in which
A is C₁-C₈-alkyl, C₁-C₈-alkoxy, phenyl or phenoxy each of which is substituted by one or more fluorine atoms, and
B is H, or
A and B together are a bridge which is substituted by one or more fluorine atoms and which, with two adjacent C atoms of the benzo ring of the formula (I), forms a five-, six- or seven-membered ring which is carbocyclic that does not contain heteroatoms or may contain heteroatoms such as O, S or N.

3. The method according to claim 1, in which
A is fluoromethyl, trifluoromethyl, 2-fluoroethyl, 2,2,2-trifluoroethyl, pentafluoroethyl, 2,2,3,3-tetrafluoropropyl, perfluorobutyl, perfluorooctyl or the corresponding alkoxy radicals, especially trifluoromethyl or trifluoromethoxy, and
B is H,
or A and B together are —OCF₂O—, —OCF₂CF₂O—, —OCHFCHFO— or —OCF₂CF₂CF₂O—.

4. The method according to claim 1, wherein at least one pigment of the formula (II) to (VIII) is used as pigment of the formula (I),

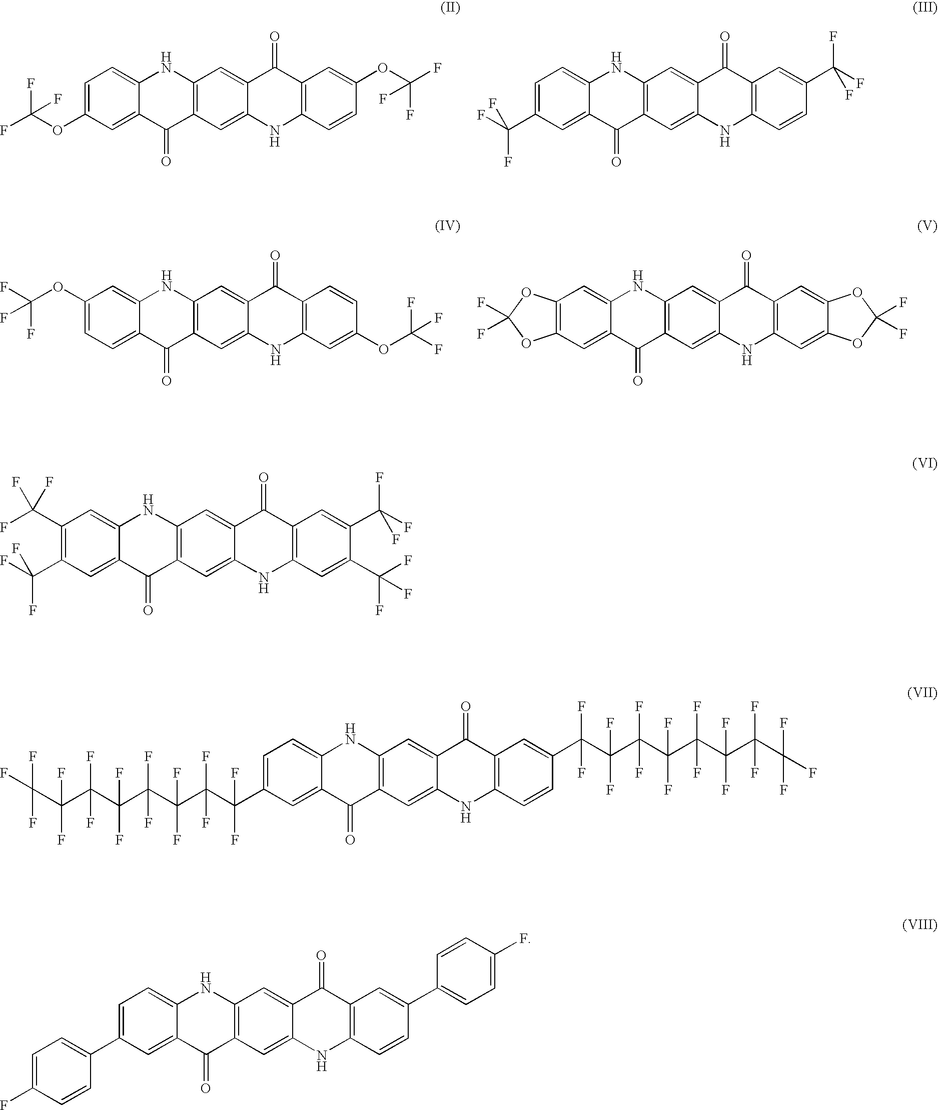

5. The method according to claim 1, wherein the pigment of the formula (I) is used in combination with other pigments.

6. The method according to claim 1, wherein the pigment of the formula (I) is used in combination with a pigment from the monoazo, disazo, laked azo, β-naphthol, Napthol AS, benzimidazolone, quinacridone, disazo condensation, azo metal complex, isoindoline and isoindolinone series, and/or with polycyclic pigments such as, for example, from the phthalocyanine, quinacridone (other than those of the formula I), perylene, perinone, thioindigo, anthraquinone, dioxazine, quinophthalone and diketopyrrolopyrrole series, and/or with laked dyes, especially Ca, Mg and Al lakes of dyes containing sulphonic or carboxylic acid groups.

7. The method according to claim 1, wherein a mixture is used comprising
at least one pigment of the formula (I)
at least one further red pigment from the group consisting of C.I. Pigment Red 122, C.I. Pigment Red 149, C.I. Pigment Red 177, C.I. Pigment Red 179, C.I. Pigment Red 254 and C.I. Pigment Violet 19.

8. The method according to claim 1, wherein a mixture is used comprising
at least one pigment of the formula (I) and
at least one yellow pigment having an absorption band in the range from 400 to 520 nm.

9. The method according to claim 1, wherein the pigment of the formula (I) has a B.E.T. surface area of 40-200 m²/g.

10. The method according to claim 1, wherein the pigment of the formula (I) possesses a dispersion harshness of 10 to 500, measured according to DIN 53775, part 7, the temperature of cold rolling being 25° C. and the temperature of hot rolling being 150° C.

11. Photoresist comprising at least one photocurable monomer, at least one photoinitiator and at least one pigment of the formula (I) as defined in claim 1.

12. Colour filter comprising at least one pigment as defined in claim 1.

13. Liquid-crystal display comprising at least one colour filter according to claim 12.

14. Compound of the formula (II) 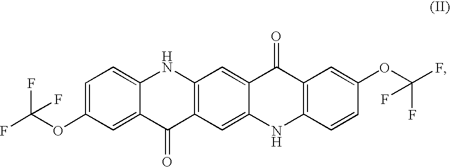
which in the X-ray diffraction diagram (Cu—K_{α} radiation) has lines at the following d values:
d: 5.30; d: 4.09; d: 3.69; d: 3.22 (A modification)

15. Compound of the formula (II) 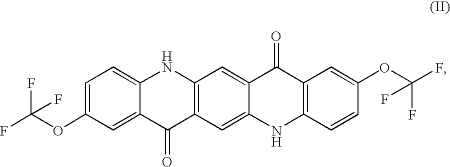
which in the X-ray diffraction diagram (Cu—K_{α} radiation) has lines at the following d values:
d: 4.22; d: 3.55; d: 3.31 (B modification).

16. Compound of the formula (IVa) 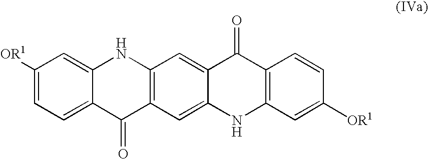
in which R¹ is a fluorine-containing aliphatic radical, especially a fluorine-containing C₁-C₈-radical, preferably fluoromethyl, trifluoromethyl, 2-fluoroethyl, 2,2,2-trifluoroethyl, pentafluoroethyl, 2,2,3,3-tetrafluoropropyl, perfluorobutyl or perfluorooctyl, especially a compound of the formula (IV)

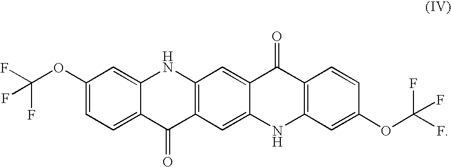

17. Compound of the formula (Va) 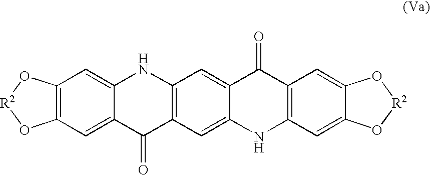
in which R² is a fluorine-substituted C₁-C₃-alkylene unit, especially —CF₂—, —CF₂CF₂—, —CHFCHF— or —CF₂CF₂CF₂—, especially a compound of the formula (V)

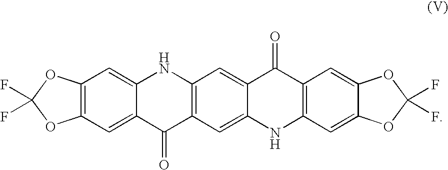

18. Mixtures comprising
at least one pigment of the formula (I)
at least one further red pigment from the group consisting of C.I. Pigment Red 122, C.I. Pigment Red 149, C.I. Pigment Red 177, C.I. Pigment Red 179, C.I. Pigment Red 254 and C.I. Pigment Violet 19.

19. Mixtures comprising
at least one pigment of the formula (I) and
at least one yellow pigment having an absorption band in the range from 400 to 520 nm.
